(19) United States
(12) Patent Application Publication
(10) Pub. No.: US 2021/0169743 A1
(43) Pub. Date: Jun. 10, 2021

(54) MEDICAL DISPOSABLE PUMPING SET

(71) Applicants: Mohammad Mehdi Nadermanesh, Shiraz (IR); Khalil Zarei, Zarghan (IR); Alireza Esteki, Shiraz (IR); Ali Zare, Shiraz (IR); Seyed Moeinoddin Bahrani, Shiraz (IR)

(72) Inventors: Mohammad Mehdi Nadermanesh, Shiraz (IR); Khalil Zarei, Zarghan (IR); Alireza Esteki, Shiraz (IR); Ali Zare, Shiraz (IR); Seyed Moeinoddin Bahrani, Shiraz (IR)

(73) Assignee: Mohammad Mehdi Nadermanesh, Shiraz (IR)

(21) Appl. No.: 17/180,795

(22) Filed: Feb. 21, 2021

Related U.S. Application Data
(60) Provisional application No. 62/980,151, filed on Feb. 22, 2020.

(51) Int. Cl. A61J 15/00; A61M 1/00

(52) U.S. Cl. CPC A61J 15/0011; A61J 15/0003; A61M 1/0019; A61M 1/0023; A61J 15/0092

(57) ABSTRACT

A system for feeding and aspirating a patient's stomach may include a feeding bag, a collection bag, and a water source interconnected with each other via a double action piston pump and further connected to a nasogastric tube via the double action piston pump. An exemplary double action piston pump may be utilized for pumping contents of the feeding bag into the patient's stomach, pumping out contents of the patient' s stomach into the collection bag, injecting water from the water source into the patient's stomach.

### CROSS-REFERENCE TO RELATED APPLICATION

This application claims the benefit of priority from pending U.S. Provisional Patent Application Ser. No. 62/980,151, filed on Feb. 22, 2020, and entitled “MEDICAL DISPOSABLE PUMPING SET,” which is incorporated herein by reference in its entirety.

### TECHNICAL FIELD

The present disclosure relates to medical pumps, particularly to double action piston pumps. More particularly, the present disclosure relates to a medical pumping assembly for feeding/aspiration process.

### BACKGROUND

Patients in intensive care units (ICUs) may not be able to eat or swallow normally due to the presence of endotracheal tube in their mouths for mechanical breathing support. In some cases, a patient's intestine may not be functioning properly, which may limit the amount of nutrition the patient receives. However, it is essential for a patient's health for them to receive adequate nutrition without disrupting the process of recovery and treatment. Therefore, it is necessary to provide a patient with an amount of nutrition that may be sufficiently absorbed by a patient's impaired gastro-intestinal tract. One possible solution may be transmitting nutrition directly into a patient's gastro-intestinal tract via a nasogastric tube. Medications may also be delivered utilizing this approach into a patient's gastro-intestinal tract to ensure full intake.

Moreover, it may be needed to remove contents of a patient's stomach, which is referred to herein as aspiration of a patient's stomach. Despite many and varied approaches to systems for aspirating a patient's stomach, many challenges still remain in providing systems for removing materials from a patient's stomach safely and reliably, without causing medical complications. Several pump apparatuses for use in conjunction with feeding and aspiration of a patient's stomach are utilized. For example, a single piston pump may be utilized for feeding and aspiration, in which a piston may be moved in a first direction to draw fluid into a cylinder, and then may be moved in a second direction to push the fluid out of the cylinder into a patient's gastro-intestinal tract via a nasogastric tube. This kind of reciprocating pump may generate pulses in fluid, which may be discontinuously fed into a nasogastric tube and subsequently into a patient's stomach. To eliminate fluid discontinuity, a multi-cylinder pump may be utilized, which may include a plurality of reciprocating pistons to provide a continuous flow of infusion fluid into a nasogastric tube connected to the multi-cylinder pump.

As mentioned above, providing a flow of infusion fluid to a patient's stomach or removing materials from a patient's stomach with a single reciprocating piston may be associated with issues, such as fluid discontinuity that should be addressed.

Delivering nutrients, fluid, and medications into a patient's stomach and aspirating a patient's stomach utilizing an infusion system that may allow for a combination of feeding and aspiration is a crucial need in the art. In currently available technologies, feeding and aspiration processes need either separate infusion systems or considerable and time-consuming changes of containers, tubes, and connectors of a system to switch its functionality. If reusable and sterile equipment is used, it is possible to wash and clean them and prepare them for another process, however, it consumes time and energy. Indeed, providing infusion systems with the capability of pumping fluid from a container into a patient's gastrointestinal tract, removing fluid from a patient's gastrointestinal tract into another container and additionally removing residual fluid or particles between a feeding cycle and an aspirating cycle with minimum effort for changing system arrangement and order would be beneficial.

There is, therefore, a need for providing a double action piston pump in an infusion system for combining feeding and aspiration processes which may be capable of executing both processes consecutively. There is further a need for a method for gastrointestinal feeding and aspiration that may allow for washing not only all infusion system ducts but also a patient's gastrointestinal tract with minimum effort.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description and the drawings.

According to one or more exemplary embodiments, the present disclosure is directed to a method for gastrointestinal feeding and aspiration. An exemplary method may include inserting a nasogastric tube inside a patient's stomach and connecting an exemplary nasogastric tube to a pumping mechanism.

An exemplary pumping mechanism may include a hollow cylinder including a first end, a second end, and an annular wall extended between the first end and the second end along a longitudinal axis of the cylinder. An exemplary hollow cylinder may further include a first port and a second port connected in fluid communication with an inner volume of the cylinder on the annular wall adjacent the first end. An exemplary hollow cylinder may further include a third port and a fourth port connected in fluid communication with the inner volume of the cylinder on the annular wall adjacent the second end.

An exemplary pumping mechanism may further include a piston that may be disposed within an exemplary hollow cylinder. An exemplary piston may divide an inner volume of an exemplary hollow cylinder into a first chamber and a second chamber. An exemplary piston may reciprocate along a longitudinal axis of an exemplary hollow cylinder in alternately a first direction and a second direction. An exemplary first direction may correspond to a direction along longitudinal axis from the first end to the second end, and an exemplary second direction may correspond to a direction along longitudinal axis from the second end to the first end. An exemplary first port and an exemplary second port may be located in an exemplary first chamber, while an exemplary third port and an exemplary fourth port may be located in an exemplary second chamber.

An exemplary pumping mechanism may further include a collection bag that may be connected in fluid communication with an exemplary first port and an exemplary third port. Fluid communication between an exemplary collection bag and an exemplary first port may be controlled by a first control valve and a third control valve, while fluid communication between an exemplary collection bag and an exemplary third port may be controlled by an exemplary first control valve and a second control valve. An exemplary second control valve may be connected in series with an exemplary first control valve, while an exemplary third control valve may be connected in series with an exemplary first control valve.

An exemplary pumping mechanism may further include a feeding bag that may be connected in fluid communication with an exemplary pumping mechanism. Fluid communication between an exemplary feeding bag and an exemplary first port controlled by a fourth control valve and an exemplary third control valve, while fluid communication between an exemplary feeding bag and an exemplary third port may be controlled by an exemplary fourth control valve and an exemplary second control valve. An exemplary second control valve may be connected in series with an exemplary fourth control valve, while an exemplary third control valve may be connected in series with an exemplary fourth control valve.

In an exemplary embodiment, connecting an exemplary nasogastric tube to an exemplary pumping mechanism may include connecting an exemplary nasogastric tube in fluid communication with an exemplary second port and an exemplary fourth port, where the fluid communication between an exemplary nasogastric tube and an exemplary second port may be controlled by a fifth control valve and the fluid communication between an exemplary nasogastric tube and an exemplary fourth port may be controlled by a sixth control valve.

In an exemplary embodiment, an exemplary method may further include aspirating a patient's stomach by pumping out contents of a patient's stomach via an exemplary nasogastric tube. Aspirating a patient's stomach may include opening an exemplary first control valve, an exemplary second control valve, and an exemplary fifth control valve responsive to an exemplary piston moving within an exemplary cylinder in an exemplary first direction, closing an exemplary third control valve, an exemplary fourth control valve, an exemplary sixth control valve, and an exemplary seventh control valve responsive to an exemplary piston moving within an exemplary cylinder in an exemplary first direction, opening an exemplary third control valve and an exemplary sixth control valve responsive to an exemplary piston moving within an exemplary cylinder in an exemplary second direction, and closing an exemplary second control valve and an exemplary fifth control valve responsive to an exemplary piston moving within an exemplary cylinder in an exemplary second direction. In an exemplary embodiment, the aspirated contents of a patient's stomach may be collected in an exemplary collection bag.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for illustration and description only and are not intended as a definition of the limits of the present disclosure. Embodiments of the present disclosure will now be described by way of example in association with the accompanying drawings in which:

FIG. 1 illustrates a method for a gastrointestinal feeding and aspiration, consistent with one or more exemplary embodiments of the present disclosure;

FIG. 2 illustrates a schematic view of a system for gastrointestinal feeding and aspiration, consistent with one or more exemplary embodiments of the present disclosure;

FIG. 3A illustrates a schematic view of a double action piston pump, consistent with one or more exemplary embodiments of the present disclosure;

FIG. 3B illustrates a schematic view of a second control valve in a first rotational position, consistent with one or more exemplary embodiments of the present disclosure;

FIG. 3C illustrates a schematic view of a second control valve in a second rotational position, consistent with one or more exemplary embodiments of the present disclosure;

FIG. 3D illustrates a schematic view of a single double action valve in an initial position, consistent with one or more exemplary embodiments of the present disclosure;

FIG. 4 illustrates a schematic view of a system for gastrointestinal feeding and aspiration connected to a nasogastric tube inserted inside a patient's stomach, consistent with one or more exemplary embodiments of the present disclosure; and

FIG. 5 illustrates a block diagram of a system for gastrointestinal feeding and aspiration, consistent with one or more exemplary embodiments of the present disclosure.

### DETAILED DESCRIPTION

The novel features which are believed to be characteristic of the present disclosure, as to its structure, organization, use and method of operation, together with further objectives and advantages thereof, will be better understood from the following discussion.

The present disclosure is directed to exemplary embodiments of a system and a method for gastrointestinal feeding and aspiration. An exemplary system for gastrointestinal feeding and aspiration may include a pumping mechanism that may be connected to a nasogastric tube. An exemplary nasogastric tube may be inserted inside a patient's stomach to allow fluid communication between an exemplary pumping mechanism and a patient's stomach, which may further allow for performing either feeding or aspiration procedures utilizing the exemplary pumping mechanism.

An exemplary system for gastrointestinal feeding and aspiration may further include one or more containers, such as a collection bag and a feeding bag. An exemplary collection bag may be utilized to collect contents of a patient's stomach. An exemplary collection bag may further be utilized to collect various types of obstructions that may be removed from a patient's stomach, such as atherosclerotic plaque, thrombus, and other types of obstructions. An exemplary feeding bag may be utilized to hold nutrients, fluids, or medications that may be fed into a patient's stomach via an exemplary pumping mechanism. An exemplary system for gastrointestinal feeding and aspiration may further include a water source that may be connected in fluid communication to an exemplary pumping mechanism and may provide the water that may be utilized for washing not only all infusion system ducts but also a patient's stomach and gastrointestinal tract.

An exemplary method for gastrointestinal feeding and aspiration may follow a standard feeding protocol. Generally, feeding a patient may be carried out in two modes, namely, a continuous mode or a bolus mode. In an exemplary continuous mode, an exemplary pumping mechanism may be set to feed a patient slowly and continuously all day long. Depending on urine output of a patient, medical staff may decide to aspirate a patient's stomach to find out their metabolism condition and change settings of an exemplary pumping mechanism if needed.

In an exemplary bolus mode, a mature patient may be fed every four hours, i.e., 6 times a day. Based on current patient feeding protocols in most countries, before each feeding session, stomach of a patient must be aspirated first to check their body metabolism. If aspirated contents of a patient's stomach is less than a predetermined threshold, aspirated contents may be returned to the stomach, along with newly prepared nutrition. However, if aspirated contents of a patient's stomach is not less than the predetermined threshold, then only the aspirated content may be pumped back to the stomach. In an exemplary embodiment, the predetermined threshold may be determined based at least in part to a patient's weight, medical condition, or a doctor's order. For example, the predetermined threshold may be 4 to 5 mL per kilogram weight of a patient.

In an exemplary embodiment, exemplary systems and methods may be configured to perform both continuous and bolus modes of feeding. In an exemplary embodiment, an exemplary pumping mechanism may be utilized without feeding and aspiration bags as a continuous positive displacement pump in other medical applications, such as a serum pump or a dialysis pump.

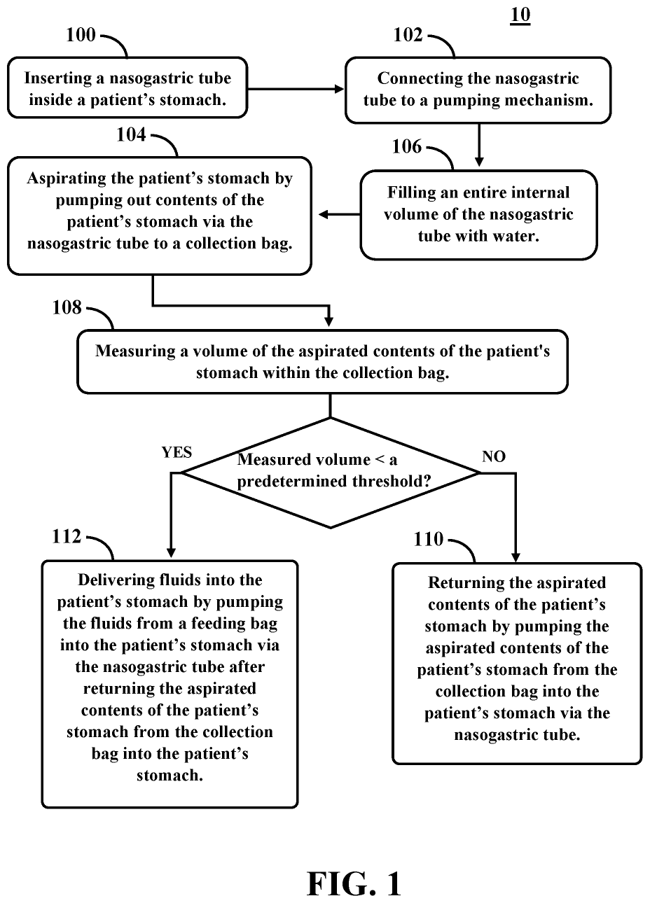

FIG. 1 illustrates a method 10 for a gastrointestinal feeding and aspiration, consistent with one or more exemplary embodiments of the present disclosure. In an exemplary embodiment, method 10 may be used for performing a bolus feeding protocol. In an exemplary embodiment, method 10 may include a step 100 of inserting a nasogastric tube inside a patient's stomach, a step 102 of connecting the nasogastric tube to a pumping mechanism, a step 104 of filling an entire internal volume of the nasogastric tube with a liquid utilizing the pumping mechanism, a step 106 of aspirating the patient's stomach by pumping out contents of the patient's stomach via the nasogastric tube into a collection bag, a step 108 of measuring a volume of the aspirated contents of the patient's stomach within the collection bag, a step 110 of returning the aspirated contents of the patient's stomach by pumping the aspirated contents of the patient's stomach from a collection bag into the patient's stomach via the nasogastric tube responsive to the measured volume equal or more than a predetermined threshold, and a step 112 of delivering nutrient/medical fluids into the patient's stomach by pumping the fluids from a feeding bag into the patient's stomach via the nasogastric tube and returning the aspirated contents of the patient's stomach by pumping the aspirated contents of the patient's stomach from a collection bag into the patient's stomach via the nasogastric tube responsive to the measured volume less than the predetermined threshold. In an exemplary embodiment, the predetermined threshold may be determined based at least in part to a patient's weight, medical condition, or a doctor's order. For example, the predetermined threshold may be 4 to 5 mL per kilogram weight of a patient. In an exemplary embodiment, all pumping out and pumping in steps may be carried out utilizing a single pumping mechanism that may allow for carrying out feeding, aspiration, and washing procedures.

In an exemplary embodiment, step 100 of inserting a nasogastric tube inside a patient's stomach may include inserting a nasogastric tube into the patient's stomach through the patient's nose. In an exemplary embodiment, inserting an exemplary nasogastric tube inside a patient's stomach may allow for gaining access to the patient's stomach and its contents. Such access to the stomach via the inserted nasogastric tube may allow for enteral feeding of a patient or draining gastric contents through the nasogastric tube. In an exemplary embodiment, step 100 of inserting a nasogastric tube inside a patient's stomach may include passing a narrow-bore tube into the patient's stomach via the nose for either short-term or medium-term nutritional support, as well as short-term or medium-term aspiration of stomach contents. In an exemplary embodiment, a nasogastric tube may be inserted and kept within a patient's stomach based at least in part on the material of that particular nasogastric tube. For example, nasogastric tubes made of silicone or polyurethane may remain in a patient's stomach for a longer period in comparison with polyvinyl chloride (PVC) tubes, due to resistance of silicone or polyurethane to heat and acidic or basic environments. In an exemplary embodiment, step 100 of inserting a nasogastric tube inside a patient's stomach may include passing a nasogastric tube via either nostril, past the pharynx, into the esophagus, and then into the stomach of the patient.

In an exemplary embodiment, step 102 of connecting the nasogastric tube to a pumping mechanism may include connecting the nasogastric tube to a pump. An exemplary pump may interconnect at least one container, a water source, and the nasogastric tube. In an exemplary embodiment, an exemplary pump of an exemplary pumping mechanism may be utilized for either feeding fluids from the at least one container into the patient's stomach via the nasogastric tube or draining the contents of the stomach via the nasogastric tube into the at least one container. In an exemplary embodiment, the pump of an exemplary pumping mechanism may further be utilized for washing the nasogastric tube, as well as the patient's stomach by pumping water into the nasogastric tube and the patient's stomach from the water source, as will be described.

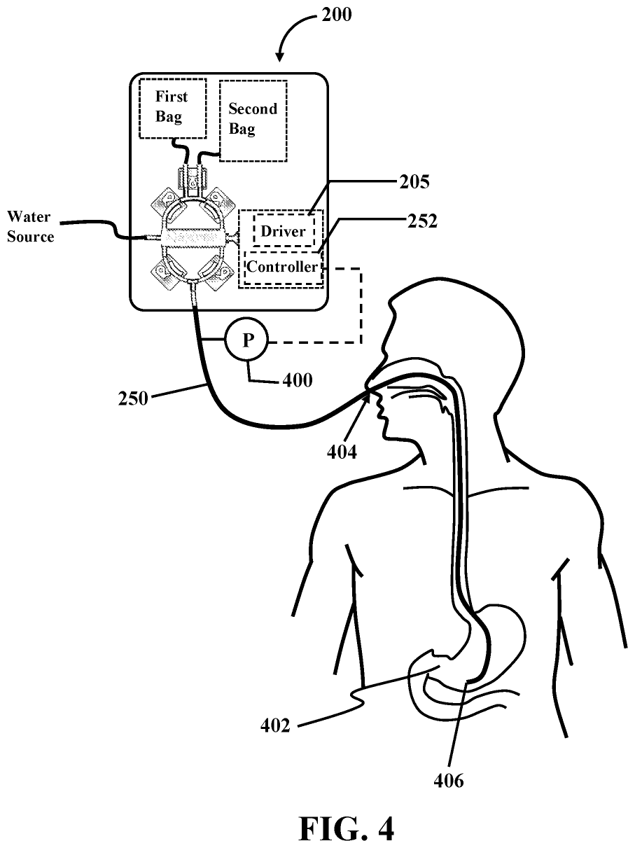

FIG. 4 illustrates a schematic view of a system 200 for gastrointestinal feeding and aspiration connected to a nasogastric tube 250 inserted inside a patient's stomach 402, consistent with one or more exemplary embodiments of the present disclosure. In an exemplary embodiment, method 10 may be implemented by a system, such as system 200. In an exemplary embodiment, step 100 of inserting a nasogastric tube inside a patient's stomach may include inserting nasogastric tube 250 into patient's stomach 402 through patient's nose 404.

In an exemplary embodiment, step 102 of connecting the nasogastric tube to a pumping mechanism may include connecting the nasogastric tube, such as nasogastric tube 250 to a pumping mechanism, such as pumping mechanism of system 200, such that one end of nasogastric tube 250 may be connected in fluid communication with system 200 and the other opposing end of nasogastric tube 250 may be positioned within patient's stomach 402. In other words, nasogastric tube 250 may provide a fluid communication line between pumping mechanism of system 200 and patient's stomach 402, which may allow for performing either feeding or aspiration procedures utilizing pumping mechanism of system 200.

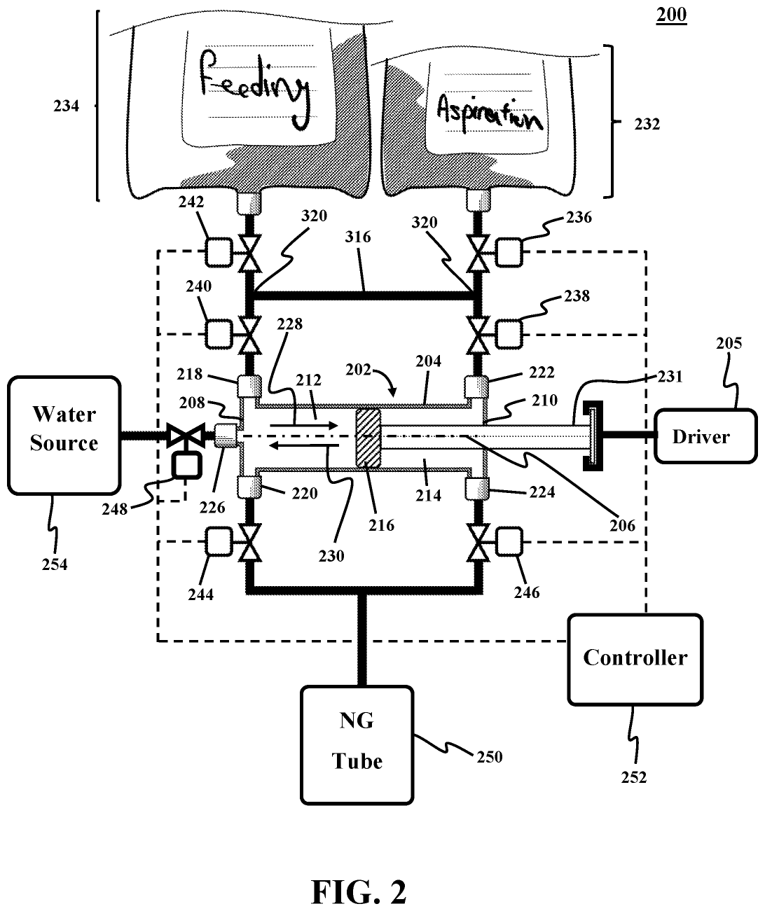

FIG. 2 illustrates a schematic view of system 200 for gastrointestinal feeding and aspiration, consistent with one or more exemplary embodiments of the present disclosure. In an exemplary embodiment, system 200 may include a double action piston pump 202. In an exemplary embodiment, double action piston pump 202 may include a hollow cylinder 204. In an exemplary embodiment, hollow cylinder 204 may include a first end 208 and a second end 210 that may be located on both sides of an annular wall of hollow cylinder 204 along a longitudinal axis 206 of hollow cylinder 204. In an exemplary embodiment, double action piston pump 202 may further include a piston 216 that may be disposed within hollow cylinder 204 and may assume a reciprocating movement within hollow cylinder 204. In an exemplary embodiment, inner volume of hollow cylinder 204 may be divided to a first chamber 212 and a second chamber 214 by piston 216. In an exemplary embodiment, piston 216 may reciprocate alternately in a first direction 228 and a second opposing direction 230, where first direction 228 may correspond to a direction along longitudinal axis 206 of hollow cylinder 204 from first end 208 to second end 210 and second direction 230 may correspond to a direction along longitudinal axis 206 of hollow cylinder 204 from second end 210 to first end 208.

In an exemplary embodiment, hollow cylinder 204 may further include a first port 218, a second port 220, a third port 222, a fourth port 224, and a fifth port 226. In an exemplary embodiment, first port 218 and second port 220 may be connected in fluid communication with inner volume of first chamber 212 adjacent first end 208. In an exemplary embodiment, third port 222 and forth port 224 may be connected in fluid communication with inner volume of second chamber 214 adjacent second end 210. In an exemplary embodiment, fifth port 226 may be connected in fluid communication with inner volume of first chamber 212 and may be located on first end 208. As used herein, an exemplary port being in fluid communication with an exemplary chamber may refer to allowing fluid to pass into or out of respective inner volumes of first chamber 212 and second chamber 214 through that exemplary port. For example, first port 218 being in fluid communication with inner volume of first chamber 212 may refer to allowing fluid to pass into or out of first chamber 212 through first port 218.

In an exemplary embodiment, system 200 for gastrointestinal feeding and aspiration may further include at least one container. For example, system 200 may include at least one container, such as a collection bag 232 and a feeding bag 234. In an exemplary embodiment, collection bag 232 may be connected in fluid communication with first port 218 and third port 222. As used herein, collection bag 232 being in fluid communication with first port 218 and third port 222 may refer to connecting collection bag 232 with first port 218 and third port 222 via tubing that may allow for fluid to pass between collection bag 232 and first port 218 and third port 222.

In an exemplary embodiment, fluid communication between collection bag 232 and first port 218 may be intercepted by a first control valve 236 and a third control valve 240. As used herein, a fluid communication between two elements being intercepted by a control valve may refer to the control valve being able to open or close the fluid communication line between the two elements. For example, first control valve 236 and a third control valve 240 intercepting fluid communication between collection bag 232 and first port 218 may refer to first control valve 236 and a third control valve 240 being able to open or close the fluid communication lines or pipes between collection bag 232 and first port 218. In an exemplary embodiment, third control valve 240 may be connected in series with first control valve 236. As used herein, third control valve 240 being connected in series with first control valve 236 may refer to an arrangement where first control valve 236 and third control valve 240 may intercept the fluid communication between collection bag 232 and first port 218, such that closing either one of first control valve 236 or third control valve 240 may disconnect the fluid communication between collection bag 232 and first port 218.

In an exemplary embodiment, fluid communication between collection bag 232 and third port 222 may be intercepted by first control valve 236 and a second control valve 238. As used herein, the fluid communication between two elements being intercepted by one or more valves may refer to utilizing one or more valves in the tubing between the two elements, such that the tubing may be opened or closed utilizing the one or more valves. For example, the fluid communication between collection bag 232 and third port 222 being intercepted by first control valve 236 and second control valve 238 may refer to the tubing between collection bag 232 and third port 222 that may allow for fluid flow between collection bag 232 and third port 222 may be opened or closed utilizing first control valve 236 and second control valve 238. In an exemplary embodiment, second control valve 238 may be connected in series with first control valve 236. As used herein, second control valve 238 being connected in series with first control valve 236 may refer to an arrangement where first control valve 236 and second control valve 238 may intercept the tubing between collection bag 232 and third port 222, such that closing either one of first control valve 236 or second control valve 238 may disconnect the fluid communication of collection bag 232 and third port 222.

In an exemplary embodiment, fluid communication between feeding bag 234 and first port 218 may be intercepted by a fourth control valve 242 and third control valve 240. In an exemplary embodiment, third control valve 240 may be connected in series with fourth control valve 242. As used herein, third control valve 240 being connected in series with fourth control valve 242 may refer to an arrangement where fourth control valve 242 and third control valve 240 may intercept the tubing between feeding bag 234 and first port 218, such that closing either one of fourth control valve 242 or third control valve 240 may disconnect the fluid communication of feeding bag 234 and first port 218.

In an exemplary embodiment, fluid communication between feeding bag 234 and third port 222 may be intercepted by fourth control valve 242 and second control valve 238. In an exemplary embodiment, second control valve 238 may be connected in series with fourth control valve 242. As used herein, second control valve 238 being connected in series with fourth control valve 242 may refer to an arrangement where fourth control valve 242 and second control valve 238 may intercept the tubing between feeding bag 234 and third port 222, such that closing either one of fourth control valve 242 or second control valve 238 may disconnect the fluid communication of feeding bag 234 and third port 222.

In an exemplary embodiment, system 200 may further include a controller 252 that may be coupled with first control valve 236, second control valve 238, third control valve 240, and fourth control valve 242. In an exemplary embodiment, controller 252 may be configured to send opening/closing commands to actuators of first control valve 236, second control valve 238, third control valve 240, and fourth control valve 242.

In an exemplary embodiment, step 102 of connecting the nasogastric tube to the pumping mechanism may include connecting nasogastric tube 250 to double action piston pump 202. In an exemplary embodiment, nasogastric tube 250 may be connected in fluid communication with second port 220. The fluid communication between nasogastric tube 250 and second port 220 may be intercepted by a fifth control valve 244. As used herein, fluid communication being intercepted by a control valve may refer to the control valve allowing for opening or closing the fluid pathway or in other words connecting or disconnecting the fluid communication. In an exemplary embodiment, nasogastric tube 250 may further be connected in fluid communication with fourth port 224. The fluid communication between nasogastric tube 250 and fourth port 224 may be intercepted by a sixth control valve 246.

In other words, in an exemplary embodiment, nasogastric tube 250 may be connected in fluid communication with first chamber 212 via second port 220 and nasogastric tube 250 may further be connected in fluid communication with second chamber 214 via fourth port 224. In an exemplary embodiment, alternate opening/closing of fifth control valve 244 and sixth control valve 246 may allow for alternately connecting/disconnecting nasogastric tube 250 with either first chamber 212 or second chamber 214. In an exemplary embodiment, controller 252 may further be configured for sending opening/closing commands to actuators of fifth control valve 244 and sixth control valve 246.

In an exemplary embodiment, system 200 may further include a water source 254 that may be connected to double action piston pump 202. In an exemplary embodiment, water source 254 may be connected in fluid communication with first chamber 212 of hollow cylinder 204. As used herein, water source 254 being in fluid communication with first chamber 212 may refer to connecting water source 254 with first chamber 212 via tubing that may allow for water to pass between water source 254 and first chamber 212. In an exemplary embodiment, water source 254 may further be connected in fluid communication with fifth port 226. The fluid communication between water source 254 and fifth port 226 may be intercepted by a seventh control valve 248. As used herein for seventh control valve 248, a control valve intercepting a fluid communication line may refer to a control valve selectively intercepting a fluid communication line by closing the fluid communication line or not intercepting by leaving the fluid communication line open.

In other words, in an exemplary embodiment, water source 254 may be connected in fluid communication with first chamber 212 via fifth port 226. In an exemplary embodiment, opening/closing of seventh control valve 248 may allow for connecting/disconnecting water source 254 with first chamber 212. In an exemplary embodiment, controller 252 may further be configured for sending opening/closing commands to an actuator of seventh control valve 248

In an exemplary embodiment, step 104 of filling an entire volume of the nasogastric tube with water may include pumping water from water source 254 into nasogastric tube 250 utilizing double action piston pump 202. In an exemplary embodiment, it may be beneficial to fill the entire volume of nasogastric tube 250 before aspirating patient's stomach 402.

Specifically, during aspirating patient's stomach 402, double action piston pump 202 may apply suction to nasogastric tube 250. Considering the flexibility of nasogastric tube 250, under a negative pressure during the aspiration process, nasogastric tube 250 may collapse inwardly due to the fact that under suction, an inner pressure of nasogastric tube 250 may be less than the pressure outside nasogastric tube 250. An inward collapse of nasogastric tube 250 may block fluid flow within nasogastric tube 250. However, in an exemplary embodiment, filling an entire volume of nasogastric tube 250 before aspirating patient's stomach 402 may prevent an inward collapse of nasogastric tube 250 due to the fact that an inner volume of nasogastric tube 250 may be filled with a fluid while negative pressure is applied by double action piston pump 202.

In an exemplary embodiment, step 104 of filling the entire volume of nasogastric tube 250 with water may include pumping water from water source 254 into nasogastric tube 250 until an entire volume of nasogastric tube 250 is filled with water. In an exemplary embodiment, pumping water from water source 254 into nasogastric tube 250 may include pumping water from water source 254 into double action piston pump 202 responsive to piston 216 moving in first direction 228. In an exemplary embodiment, pumping water from water source 254 into double action piston pump 202 may include closing third control valve 240, fourth control valve 242, fifth control valve 244, and sixth control valve 246 and opening first control valve 236, second control valve 238, and seventh control valve 248 responsive to piston 216 moving within hollow cylinder 204 in first direction 228. In an exemplary embodiment, pumping water from water source 254 into double action piston pump 202 may further include closing seventh control valve 248 and opening fifth control valve 244 responsive to piston 216 moving within hollow cylinder 204 in second direction 230.

As used herein, pumping water from water source 254 into double action piston pump 202 may refer to pumping water from water source 254 via fifth port 226 through seventh control valve 248 into first chamber 212 and pumping contents of second chamber 214 via third port 222 through second control valve 238 and first control valve 236 into collection bag 232. Furthermore, pumping water from double action piston pump 202 into nasogastric tube 250 may refer to pumping water from first chamber 212 via second port 220 through fifth control valve 244 into nasogastric tube 250.

In an exemplary embodiment, step 106 of aspirating patient's stomach 402 may include pumping out contents of patient's stomach 402. In an exemplary embodiment, step 106 of aspirating patient's stomach 402 may further include pumping out contents of patient's stomach 402 via nasogastric tube 250 into collection bag 232 utilizing double action piston pump 202.

In an exemplary embodiment, pumping out contents of patient's stomach 402 into collection bag 232 may include opening first control valve 236, second control valve 238, and fifth control valve 244 and closing third control valve 240, fourth control valve 242, sixth control valve 246, and seventh control valve 248 responsive to piston 216 moving within hollow cylinder 204 in first direction 228. In an exemplary embodiment, pumping out contents of patient's stomach 402 into collection bag 232 may further include opening third control valve 240 and sixth control valve 246 and closing second control valve 238 and fifth control valve 244 responsive to piston 216 moving within hollow cylinder 204 in second direction 230. In other words, by opening and closing specific control valves as discussed above during the reciprocating movement of piston 216 back and forth in first direction 228 and second direction 230, a continuous flow of fluids from patient's stomach 402 into collection bag 232 may be established. Specifically, in response to piston 216 moving in first direction 228, controller 252 may send an opening signal to the actuators of first control valve 236, second control valve 238, and fifth control valve 244 and a closing signal to the actuators of third control valve 240, fourth control valve 242, sixth control valve 246, and seventh control valve 248. This way, in each reciprocation of piston 216, when piston 216 moves in first direction 228, the contents of patient's stomach 402 may be pumped into first chamber 212 via second port 220, while the contents of patient's stomach 402 already pumped into second chamber 214 may be simultaneously pumped out into collection bag 232 via third port 222. On the other hand, in response to piston 216 moving in second direction 230, controller 252 may send an opening signal to the actuators of third control valve 240 and sixth control valve 246 and a closing signal to the actuators of second control valve 238 and fifth control valve 244. This way, in each reciprocation of piston 216, when piston 216 moves in second direction 230, the contents of patient's stomach 402 may be pumped into second chamber 214 via third port 222, while the contents of patient's stomach 402 already pumped into first chamber 212 may be simultaneously pumped out into collection bag 232 via first port 218. In an exemplary embodiment, seventh control valve 248 may include a spring-loaded one-way valve that may only allow fluid flow out of water source 254. Such configuration of seventh control valve 248 may simplify the design of the system since there is no need for controller 252 to send opening/closing signals to seventh control valve 248.

In an exemplary embodiment, step 108 of measuring a volume of the aspirated contents of the patient's stomach within the collection bag may include utilizing volume scales marked on collection bag 232 to determine how much fluid is gathered within collection bag 232. In an exemplary embodiment, other methods such as weighing collection bag 232 or utilizing flow sensors may further be used to measure a volume of the aspirated contents of the patient's stomach within the collection bag. In an exemplary embodiment, a volume of the aspirated contents of the patient's stomach within the collection bag more than a predetermined threshold, may indicate that a patient's stomach has not functioned properly, and the food has not been digested well. Consequently, as will be discussed in the following paragraphs, if the measured volume is equal or more than the predetermined threshold for a patient, the aspirated contents of that patient's stomach must be returned into the stomach to continue the process of digestion. As mentioned before, in an exemplary embodiment, the predetermined threshold may be 4-5 mL per kilogram weight of a patient, in the absence of secondary considerations, like a special medical condition or a doctor's orders.

In an exemplary embodiment, step 110 of returning the aspirated contents of patient's stomach 402 into patient's stomach 402 may include pumping aspirated contents of patient's stomach 402 from collection bag 232 via nasogastric tube 250 into patient's stomach 402 utilizing double action piston pump 202.

In an exemplary embodiment, pumping aspirated contents of patient's stomach 402 from collection bag 232 into patient's stomach 402 may include opening first control valve 236, third control valve 240, and sixth control valve 246 and closing second control valve 238, fourth control valve 242, fifth control valve 244, and seventh control valve 248 responsive to piston 216 moving within hollow cylinder 204 in first direction 228. In an exemplary embodiment, pumping aspirated contents of patient's stomach 402 from collection bag 232 into patient's stomach 402 may further include opening second control valve 238 and fifth control valve 244 and closing third control valve 240 and sixth control valve 246 responsive to piston 216 moving within hollow cylinder 204 in second direction 230.

In other words, by opening and closing specific control valves as discussed above during the reciprocating movement of piston 216 back and forth in first direction 228 and second direction 230, a continuous flow of fluids from collection bag 232 into patient's stomach 402 may be established. Specifically, in response to piston 216 moving in first direction 228, controller 252 may send an opening signal to the actuators of first control valve 236, third control valve 240, and sixth control valve 246 and a closing signal to the actuators of second control valve 238, fourth control valve 242, fifth control valve 244, and seventh control valve 248. This way, in each reciprocation of piston 216, when piston 216 moves in first direction 228, the contents of collection bag 232 may be pumped into first chamber 212 via first port 218, while the contents of collection bag 232 already pumped into second chamber 214 may be simultaneously pumped out into patient's stomach 402 via fourth port 224. On the other hand, in response to piston 216 moving in second direction 230, controller 252 may send an opening signal to the actuators of second control valve 238 and fifth control valve 244 and a closing signal to the actuators of third control valve 240 and sixth control valve 246. This way, in each reciprocation of piston 216, when piston 216 moves in second direction 230, the contents of collection bag 232 may be pumped into second chamber 214 via third port 222, while the contents of collection bag 232 already pumped into first chamber 212 may be simultaneously pumped out into patient's stomach 402 via second port 220.

In an exemplary embodiment, as mentioned before, responsive to the measured volume being equal or more than the predetermined threshold within collection bag 232, aspirated contents may be pumped back from collection bag 232 into patient's stomach 402, as discussed in the preceding paragraph. In an exemplary embodiment, if the measured volume of the aspirated content within collection bag 232 is less than the predetermined threshold, it may indicate that the entire food or a portion of the food fed to the patient in a previous feeding process has been completely or partially digested. In this case, in addition to returning the aspirated contents back into patient's stomach 402, newly prepared nutritious fluids may further be fed into patient's stomach, as will be discussed in the following paragraphs.

In an exemplary embodiment, step 112 of delivering fluids into patient's stomach 402 may include pumping fluids from feeding bag 234 into patient's stomach 402. In an exemplary embodiment, step 112 of delivering fluids into patient's stomach 402 may further include pumping fluids from feeding bag 234 via nasogastric tube 250 into patient's stomach 402 utilizing double action piston pump 202.

In an exemplary embodiment, pumping fluids from feeding bag 234 into patient's stomach 402 may include opening third control valve 240, fourth control valve 242, and sixth control valve 246 and closing first control valve 236, second control valve 238, fifth control valve 244, and seventh control valve 248 responsive to piston 216 moving within hollow cylinder 204 in first direction 228. In an exemplary embodiment, pumping fluids from feeding bag 234 into patient's stomach 402 may further include opening second control valve 238 and fifth control valve 244 and closing third control valve 240 and sixth control valve 246 responsive to piston 216 moving within hollow cylinder 204 in second direction 230.

In other words, by opening and closing specific control valves as discussed above during the reciprocating movement of piston 216 back and forth in first direction 228 and second direction 230, a continuous flow of fluids from feeding bag 234 into patient's stomach 402 may be established. Specifically, in response to piston 216 moving in first direction 228, controller 252 may send an opening signal to the actuators of third control valve 240, fourth control valve 242, and sixth control valve 246 and a closing signal to the actuators of first control valve 236, second control valve 238, and fifth control valve 244. This way, in each reciprocation of piston 216, when piston 216 moves in first direction 228, the contents of feeding bag 234 may be pumped into first chamber 212 via first port 218, while the contents of feeding bag 234 already pumped into second chamber 214 may be simultaneously pumped out into patient's stomach 402 via fourth port 224. On the other hand, in response to piston 216 moving in second direction 230, controller 252 may send an opening signal to the actuators of second control valve 238 and fifth control valve 244 and a closing signal to the actuators of third control valve 240 and sixth control valve 246. This way, in each reciprocation of piston 216, when piston 216 moves in second direction 230, the contents of feeding bag 234 may be pumped into second chamber 214 via third port 222, while the contents of feeding bag 234 already pumped into first chamber 212 may be simultaneously pumped out into patient's stomach 402 via second port 220.

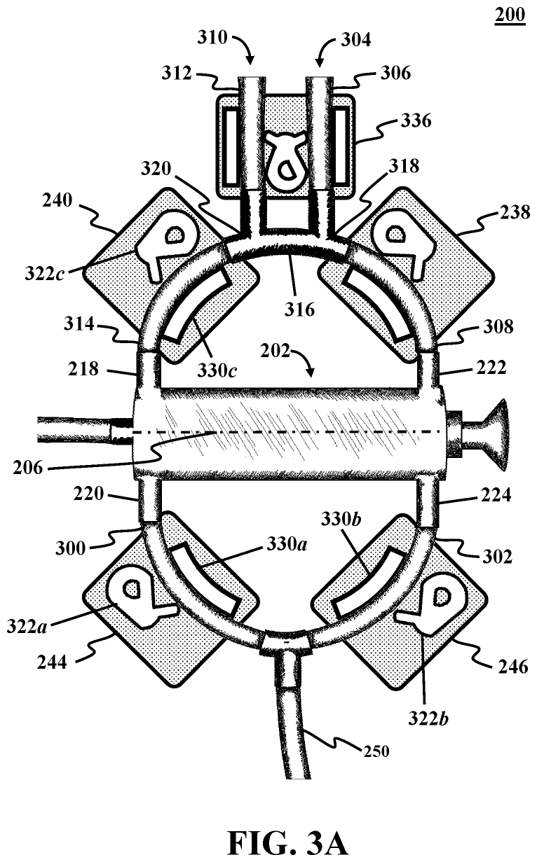

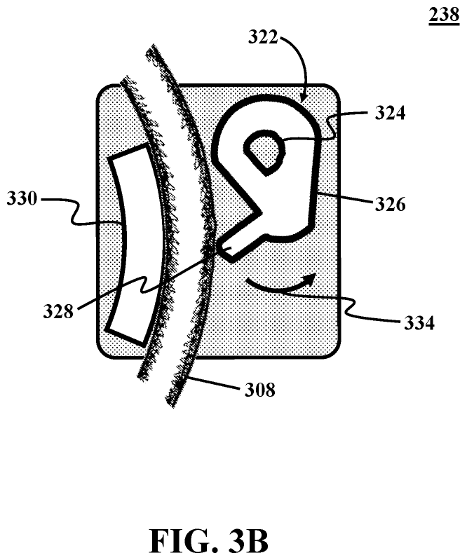

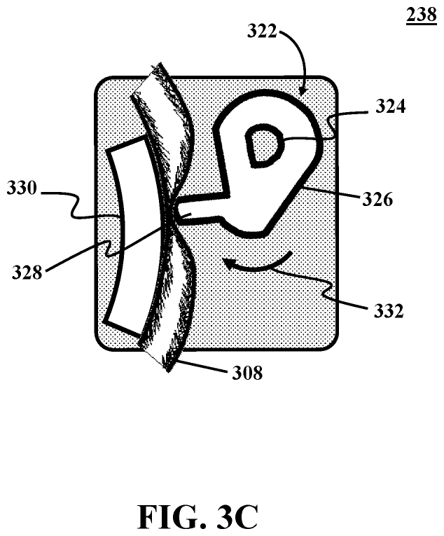

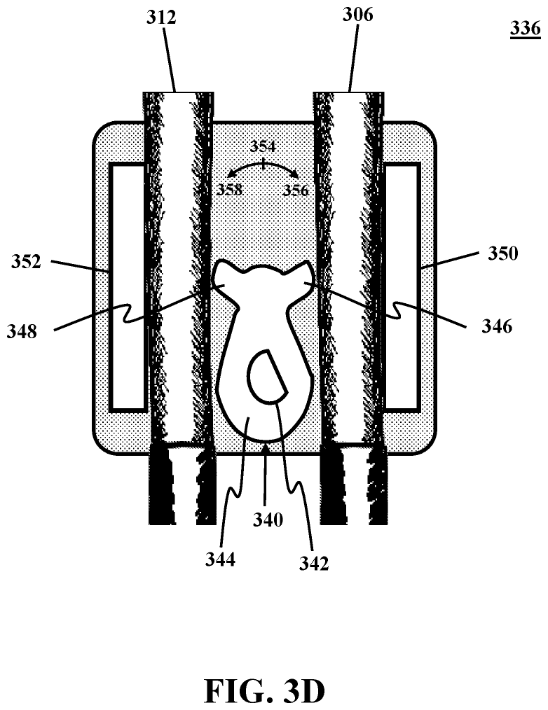

FIG. 3A illustrates a schematic view of double action piston pump 202, consistent with one or more exemplary embodiments of the present disclosure. FIG. 3B illustrates a schematic view of second control valve 238 in a first rotational position, consistent with one or more exemplary embodiments of the present disclosure. FIG. 3C illustrates a schematic view of second control valve 238 in second rotational position, consistent with one or more exemplary embodiments of the present disclosure. FIG. 3D illustrates a schematic view of single double action valve 336 in an initial position 354, consistent with one or more exemplary embodiments of the present disclosure.

In an exemplary embodiment, system 200 for gastrointestinal feeding and aspiration may include first control valve 236, second control valve 238, third control valve 240, fourth control valve 242, fifth control valve 244, sixth control valve 246, and seventh control valve 248 that may be structurally similar to each other. For example, second control valve 238 may include a motor (not illustrated) and a cam 322 that may be coupled to the motor via a shaft 324. In an exemplary embodiment, cam 322 may include a main body 326 and an extended lip 328 that may protrude from main body 326 along a plane perpendicular to a longitudinal axis of shaft 324. In an exemplary embodiment, the motor may be configured to drive a rotational movement of cam 322 about a longitudinal axis of shaft 324 in response to opening/closing commands that may be received from controller 252.

In an exemplary embodiment, second control valve 238 may further include a seat 330 that may be mounted adjacent cam 322 with a gap between seat 330 and main body 326 of cam 322 along an axis perpendicular to a longitudinal axis of shaft 324. In an exemplary embodiment, the motor (not illustrated) may further be configured to drive a rotational movement of cam 322 between a first rotational position (illustrated in FIG. 3B) and second rotational position (illustrated in FIG. 3C). In an exemplary embodiment, second control valve 238 may be closed by rotating cam 322 into second rotational position in a direction shown by arrow 334. As used herein, opening second control valve 238 may refer to opening a gap between seat 330 and main body 326. In other words, in an exemplary embodiment, extended lip 328 may not be positioned between main body 326 of cam 322 and seat 330 along an axis perpendicular to a longitudinal axis of shaft 324. In an exemplary embodiment, the motor may further be configured to drive a rotational movement of cam 322 from second rotational position to first rotational position in a direction shown by arrow 334 in response to an opening command that may be received from controller 252.

In an exemplary embodiment, second control valve 238 may be opened by rotating cam 322 into first rotational position. As used herein, closing second control valve 238 may refer to closing a gap between seat 330 and main body 326. In other words, in an exemplary embodiment, extended lip 328 may be positioned between main body 326 of cam 322 and seat 330 along an axis perpendicular to a longitudinal axis of shaft 324. In an exemplary embodiment, the motor may further be configured to drive a rotational movement of cam 322 from the first rotational position to the second rotational position in a direction shown by arrow 332 in response to closing command that may be received from controller 252. In an exemplary embodiment, first control valve 236, third control valve 240, fourth control valve 242, fifth control valve 244, sixth control valve 246, and seventh control valve 248 may have similar structures, which are not described separately, for simplicity.

In an exemplary embodiment, nasogastric tube 250 may be connected to second port 220 of double action piston pump 202 via a first flexible tube 300. In an exemplary embodiment, first flexible tube 300 may be positioned in a gap between seat 330a and cam 322a of fifth control valve 244. In an exemplary embodiment, nasogastric tube 250 may be connected to forth port 224 of double action piston pump 202 via a second flexible tube 302. In other words, in an exemplary embodiment, second flexible tube 302 may be positioned in a gap between seat 330b and cam 322b of sixth control valve 246.

In an exemplary embodiment, collection bag 232 may be connected to double action piston pump 202. In an exemplary embodiment, collection bag 232 may be connected to third port 222 of double action piston pump 202 via a third flexible tube 304. In an exemplary embodiment, third flexible tube 304 may include a first portion 306 of third flexible tube 304. In an exemplary embodiment, first portion 306 of third flexible tube 304 may be positioned in a gap between the seat and the cam of first control valve 236. In an exemplary embodiment, third flexible tube 304 may further include a second portion 308 of third flexible tube 304. In an exemplary embodiment, second portion 308 of third flexible tube 304 may be positioned in the gap between the seat and the cam of second control valve 238.

In an exemplary embodiment, feeding bag 234 may be connected to double action piston pump 202. In an exemplary embodiment, feeding bag 234 may be connected to first port 218 of double action piston pump 202 via a fourth flexible tube 310. In an exemplary embodiment, fourth flexible tube 310 may include a first portion 312 of fourth flexible tube 310. In an exemplary embodiment, first portion 312 of fourth flexible tube 310 may be positioned in a gap between the seat and the cam of fourth control valve 242. In an exemplary embodiment, fourth flexible tube 310 may further include a second portion 314 of fourth flexible tube 310. In an exemplary embodiment, second portion 314 of fourth flexible tube 310 may be positioned in the gap between seat 330c and cam 322c of third control valve 240.

In an exemplary embodiment, third flexible tube 304 and fourth flexible tube 310 may be interconnected via an interconnect tube 316. In an exemplary embodiment, interconnect tube 316 may include a first end 318 and a second opposing end 320. In an exemplary embodiment, first end 318 of interconnect tube 316 may be connected in fluid communication with third flexible tube 304 at a point along third flexible tube 304 between first control valve 236 and second control valve 238. In an exemplary embodiment, second opposite end 320 of interconnect tube 316 may be connected in fluid communication with fourth flexible tube 310 at a point along forth flexible tube 310 between fourth control valve 242 and third control valve 240.

In an exemplary embodiment, system 200 for gastrointestinal feeding and aspiration may include single double action valve 336. In an exemplary embodiment, single double action valve 336 may be utilized as an alternative to first control valve 236 and fourth control valve 242. In an exemplary embodiment, single double action valve 336 may intercept first portion 306 of third flexible tube 304 as an alternative to first control valve 236. In an exemplary embodiment, single double action valve 336 may intercept first portion 312 of forth flexible tube 312 as an alternative to fourth control valve 242.

In an exemplary embodiment, changing the status of single double action valve 336 may be utilized as an alternative to opening/closing of either first control valve 236 or fourth control valve 242. In an exemplary embodiment, single double action valve 336 may include a motor (not illustrated) and a cam 340 that may be coupled to the motor via a shaft 342. In an exemplary embodiment, cam 340 may include a main body 344 and a first extended lip 346 and a second extended lip 348 that may protrude from main body 344 along a plane perpendicular to a longitudinal axis of shaft 342. In an exemplary embodiment, first extended lip 346 may be attached to first side of main body 344 and second extended lip 348 may be attached to second opposite side of main body 344 along an axis perpendicular to a longitudinal axis of shaft 342. In an exemplary embodiment, the motor may be configured to drive a rotational movement of cam 340 about a longitudinal axis of shaft 342 in response to opening/closing commands that may be received from controller 252.

In an exemplary embodiment, single double action valve 336 may further include a first seat 350. In an exemplary embodiment, first seat 350 may be mounted on a first side of cam 340 with a gap between first seat 350 and main body 344 of cam 340 along an axis perpendicular to a longitudinal axis of shaft 342. In other words, in an exemplary embodiment, first portion of third flexible tube 306 may be positioned between first seat 350 and first extended lip 346 at first side of main body 344. In an exemplary embodiment, single double action valve 336 may further include a second seat 352. In an exemplary embodiment, second seat 352 may be mounted on a second opposite side of cam 340 with a gap between seat 352 and main body 344 of cam 340 along an axis perpendicular to a longitudinal axis of shaft 342. In other words, in an exemplary embodiment, first portion of forth flexible tube 312 may be positioned between second seat 352 and second extended lip 348 at second opposite side of main body 344.

In an exemplary embodiment, the motor may further be configured to drive rotational movement of cam 340 about a longitudinal axis of shaft 342 from initial position 354 to either a first position 356 or a second position 358. As used herein, initial position 354 may refer to a position where first extended lip 346 may be positioned adjacent an outer surface of first portion of third flexible tube 340. Furthermore, initial position 354 may further refer to a position where second extended lip 348 may be positioned adjacent an outer surface of first portion of forth flexible tube 360. As used herein, in an exemplary embodiment, first position 356 may refer to a position where first extended lip 346 may be positioned between first seat 350 and main body 344 that may press shut third flexible tube 306. As used herein, in an exemplary embodiment, second position 358 may refer to a position where second extended lip 348 may be positioned between second seat 352 and main body 344 that may press shut forth flexible tube 312.

In an exemplary embodiment, such configuration of control valves within system 200, where control valves are not in contact with fluids flowing through tubes of system 200 may allow for utilizing system 200 in applications, where contamination of the fluids are especially important. For example, when system 200 is utilized for feeding a patient, a user does not want any type of contamination to come in contact with medical or nutritious fluids flowing within system 200. In an exemplary embodiment, control valves of system 200 are designed such that the control valves may only contact an outer surface of the tubes, as was discussed in preceding paragraphs, and, therefore, no contact is made between the fluids and the control valves. Furthermore, such configuration of control valves may further allow for easily removing all the single-use tubes of system, while the control valves themselves do not need to be changed, due to their separate configuration from the tubes. For example, second portion 308 of third flexible tube 304 may be easily detached from second control valve 238 by pulling second portion 308 out of the gap between seat 330 and cam 322. Furthermore, as evident in FIGS. 3B and 3C, closing and opening of second portion 308 utilizing second control valve 238 may be carried out without second control valve 238 or any of its components coming into contact with a fluid that may be flowing within second portion 308.

In an exemplary embodiment, system 200 may further include a driver 205 that may be coupled with piston 216 via piston shaft 231. Driver 205 may be a motor that may be configured to drive a reciprocating movement of piston 216 within hollow cylinder 204.

In an exemplary embodiment, system 200 may further include a pressure transducer 400 that may be coupled with nasogastric tube 250 and may be configured to measure the fluid pressure within nasogastric tube 250. In an exemplary embodiment, pressure transducer 400 may further be configured to send the measured pressure to controller 252. During aspiration of patient's stomach 402 utilizing system 200, when contents of patient's stomach 402 is completely aspirated, due to the suction that is being applied by system 200, a distal end 406 of nasogastric tube 250 may grab a portion of an internal surface of patient's stomach 402 and cause a scar or unwanted damage to the internal surface of patient's stomach 402, which may lead to bleeding within patient's stomach 402. In an exemplary embodiment, responsive to the contents of patient's stomach 402 being completely drawn out of patient's stomach 402 and distal end 406 getting caught on the internal surface of distal end 406 due to suction provided by system 200, pressure transducer 400 may sense a sudden decrease in the pressure within nasogastric tube 250. In an exemplary embodiment, such sudden decrease in the pressure may be transmitted to controller 252, where controller 252 may be configured to shut driver 205 off to avoid any damage to the internal surface of patient's stomach 402.

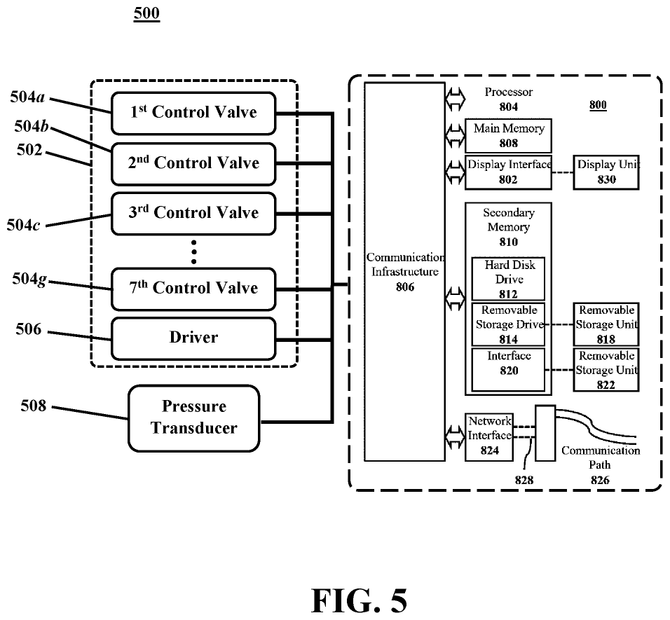

FIG. 5 illustrates a block diagram of a system 500 for gastrointestinal feeding and aspiration, consistent with one or more exemplary embodiments of the present disclosure. In an exemplary embodiment, system 500 for gastrointestinal feeding and aspiration may be similar to system 200 for gastrointestinal feeding and aspiration.

In an exemplary embodiment, system 500 may include a controller 800 that may be similar to controller 252 that may be coupled with a pumping mechanism 502 that may be similar to pumping mechanism of system 200. In an exemplary embodiment, pumping mechanism 502 may include a driver 506 similar to driver 205 that may be coupled with a double action piston pump similar to double action piston pump 202 and may be configured to drive a reciprocating motion of the double action pump. In an exemplary embodiment, pumping mechanism 502 may further include seven control valves (504a-504g) that may be similar to respective first control valve 236, second control valve 238, third control valve 240, fourth control valve 242, fifth control valve 244, sixth control valve 246, and seventh control valve 248. In an exemplary embodiment, controller 800 may be coupled to seven control valves (504a-504g) and may be configured to send opening/closing commands to actuators of seven control valves (504a-504g) to perform feeding and aspirating processes similar to method 10 for gastrointestinal feeding and aspiration. In an exemplary embodiment, controller 800 may further be coupled to a pressure transducer 508 that may be similar to pressure transducer 400 and may be configured to receive measured pressure of a nasogastric tube similar to nasogastric tube 250. In an exemplary embodiment, controller 800 may further be configured to send a shutdown command to driver 506 in response to the received pressure measurement from pressure transducer 508 showing a sudden decrease.

In an exemplary embodiment, controller 800 may be implemented as a computer system, in which an embodiment of the present disclosure, or portions thereof, may be implemented as computer-readable code, consistent with exemplary embodiments of the present disclosure. For example, method 10 for gastrointestinal feeding and aspiration may be implemented in controller 800 using hardware, software, firmware, tangible computer-readable media having instructions stored thereon, or a combination thereof and may be implemented in one or more computer systems or other processing systems.

If programmable logic is used, such logic may execute on a commercially available processing platform or a special purpose device. One ordinary skill in the art may appreciate that an embodiment of the disclosed subject matter may be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the disclosure is described in terms of this example controller 800. After reading this description, it will become apparent to a person skilled in the relevant art how to implement the disclosure using other computer systems and/or computer architectures. Although operations may be described as a sequential process, some of the operations may be performed in parallel, concurrently, and/or in a distributed environment, and with program code stored locally or remotely for access by single or multi-processor machines. Also, in some embodiments, the order of operations may be rearranged without departing from the spirit of the disclosed subject matter.

Processor device 804 may be a special purpose or a general-purpose processor device. As will be appreciated by persons skilled in the relevant art, processor device 804 may also be a single processor in a multi-core/multiprocessor system, such system operating alone, or in a cluster of computing devices operating in a cluster or server farm. Processor device 804 may be connected to a communication infrastructure 806, for example, a bus, message queue, network, or multi-core message-passing scheme.

In an exemplary embodiment, controller 800 may include a display interface 802, for example, a video connector, to transfer data to a display unit 830, for example, a monitor. Controller 800 may also include a main memory 808, for example, random access memory (RAM), and may also include a secondary memory 810. Secondary memory 810 may include, for example, a hard disk drive 812, and a removable storage drive 814. Removable storage drive 814 may include a floppy disk drive, a magnetic tape drive, an optical disk drive, a flash memory, or the like. Removable storage drive 814 may read from and/or write to a removable storage unit 818 in a well-known manner. Removable storage unit 818 may include a floppy disk, a magnetic tape, an optical disk, etc., which may be read by and written to by removable storage drive 814. As will be appreciated by persons skilled in the relevant art, removable storage unit 818 may include a computer-usable storage medium having stored therein computer software and/or data.

In alternative implementations, secondary memory 810 may include other similar means for allowing computer programs or other instructions to be loaded into controller 800. Such means may include, for example, a removable storage unit 822 and an interface 820. Examples of such means may include a program cartridge and cartridge interface (such as that found in video game devices), a removable memory chip (such as an EPROM, or PROM) and associated socket, and other removable storage units 822 and interfaces 820 which allow software and data to be transferred from removable storage unit 822 to controller 800.

Controller 800 may also include a communications interface 824. Communications interface 824 allows software and data to be transferred between controller 800 and external devices. Communications interface 824 may include a modem, a network interface (such as an Ethernet card), a communications port, a PCMCIA slot, and card, or the like. Software and data transferred via communications interface 824 may be in the form of signals, which may be electronic, electromagnetic, optical, or other signals capable of being received by communications interface 824. These signals may be provided to communications interface 824 via a communications path 826. Communications path 826 carries signals and may be implemented using wire or cable, fiber optics, a phone line, a cellular phone link, an RF link, or other communications channels.

In an exemplary embodiment, operations in method 10 may be stored in main memory 808 and/or secondary memory 810 as computer programs (also called computer control logic). Computer programs may also be received via communications interface 824. Such computer programs, when executed, enable controller 800 to implement different embodiments of the present disclosure as discussed herein. In particular, the computer programs, when executed, enable processor device 804 to implement the processes of the present disclosure, such as the operations in method 10. Accordingly, such computer programs represent controllers of controller 800. The software may be stored in a computer program product and loaded into controller 800 using removable storage drive 814, interface 820, and hard disk drive 812, or communications interface 824.

Embodiments of the present disclosure also may be directed to computer program products including software stored on any computer useable medium. Such software, when executed in one or more data processing devices, causes a data processing device to operate as described herein. An embodiment of the present disclosure may employ any computer useable or readable medium. Examples of computer useable mediums include, but are not limited to, primary storage devices (e.g., any type of random-access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps.

Moreover, the word “substantially” when used with an adjective or adverb is intended to enhance the scope of the particular characteristic; e.g., substantially planar is intended to mean planar, nearly planar and/or exhibiting characteristics associated with a planar element.

Further use of relative terms such as “vertical”, “horizontal”, “up”, “down”, and “side-to-side” are used in a relative sense to the normal orientation of the apparatus.

What is claimed is:

1. A method for gastrointestinal feeding and aspiration, the method comprising:
inserting a nasogastric tube inside a patient's stomach;
connecting the nasogastric tube to a pumping mechanism, the pumping mechanism comprising:
a hollow cylinder comprising a first end, a second end, and an annular wall extended between the first end and the second end along a longitudinal axis of the cylinder, the hollow cylinder further comprising a first port and a second port connected in fluid communication with an inner volume of the cylinder on the annular wall adjacent the first end, the hollow cylinder further comprising a third port and a fourth port connected in fluid communication with the inner volume of the cylinder on the annular wall adjacent the second end; and
a piston disposed within the cylinder, the piston dividing the inner volume of the cylinder into a first chamber and a second chamber, the piston reciprocating along the longitudinal axis in alternately a first direction and a second direction, the first direction corresponding to a direction along longitudinal axis from the first end to the second end, the second direction corresponding to a direction along longitudinal axis from the second end to the first end, the first port and the second port located in the first chamber, the third port and the fourth port located in the second chamber;
a collection bag connected in fluid communication with the first port and the third port, the fluid communication between the collection bag and the first port controlled by a first control valve and a third control valve, the fluid communication between the collection bag and the third port controlled by the first control valve and a second control valve, the second control valve connected in series with the first control valve, the third control valve connected in series with the first control valve;
a feeding bag connected in fluid communication with the first port and the third port, the fluid communication between the feeding bag and the first port controlled by a fourth control valve and the third control valve, the fluid communication between the feeding bag and the third port controlled by the fourth control valve and the second control valve, the second control valve connected in series with the fourth control valve, the third control valve connected in series with the fourth control valve,
wherein, connecting the nasogastric tube to the pumping mechanism comprises connecting the nasogastric tube in fluid communication with the second port and the fourth port, the fluid communication between the nasogastric tube and the second port controlled by a fifth control valve, the fluid communication between the nasogastric tube and the fourth port controlled by a sixth control valve; and
aspirating the patient's stomach by pumping out contents of the patient's stomach via the nasogastric tube, aspirating the patient's stomach comprising:
opening the first control valve, the second control valve, and the fifth control valve responsive to the piston moving within the cylinder in the first direction;
closing the third control valve, the fourth control valve, the sixth control valve, and the seventh control valve responsive to the piston moving within the cylinder in the first direction;
opening the third control valve and the sixth control valve responsive to the piston moving within the cylinder in the second direction; and
closing the second control valve and the fifth control valve responsive to the piston moving within the cylinder in the second direction,
wherein the aspirated contents of the patient's stomach is collected in the collection bag.

2. The method of claim 1, further comprising returning the aspirated contents of the patient's stomach into the patient's stomach responsive to a volume of the aspirated contents within the collection bag being equal to or more than a predetermined threshold, wherein by returning the aspirated contents of the patient's stomach into the patient's stomach comprises pumping the aspirated contents of the patient's stomach from the collection bag into the patient's stomach via the nasogastric tube.

3. The method of claim 2, wherein returning the aspirated contents further comprises:
opening the first control valve, the third control valve, and the sixth control valve responsive to the piston moving within the cylinder in the first direction;
closing the second control valve, the fourth control valve, the fifth control valve and the seventh control valve responsive to the piston moving within the cylinder in the first direction;
opening the second control valve and the fifth control valve responsive to the piston moving within the cylinder in the second direction; and
closing the third control valve and the sixth control valve responsive to the piston moving within the cylinder in the second direction.

4. The method according to claim 2, wherein the predetermined threshold comprises 4-5 mL per kilogram weight of the patient.

5. The method of claim 1, further comprising:
delivering medical/nutritious fluids into the patient's stomach responsive to a volume of the aspirated contents within the collection bag being less than a predetermined ratio, delivering medical/nutritious fluids into the patient's stomach comprising pumping the medical/nutritious fluids from the feeding bag into the patient's stomach via the nasogastric tube; and
returning the aspirated contents of the patient's stomach into the patient's stomach responsive to a volume of the aspirated contents within the collection bag being less than the predetermined ratio, wherein by returning the aspirated contents of the patient's stomach into the patient's stomach comprises pumping the aspirated contents of the patient's stomach from the collection bag into the patient's stomach via the nasogastric tube.

6. The method of claim 5, wherein delivering medical/nutritious fluids into the patient's stomach comprises:
opening the third control valve, the fourth control valve, and the sixth control valve responsive to the piston moving within the cylinder in the first direction;
closing the first control valve, the second control valve, the fifth control valve, and the seventh control valve responsive to the piston moving within the cylinder in the first direction;
opening the second control valve and the fifth control valve responsive to the piston moving within the cylinder in the second direction; and
closing the third control valve and the sixth control valve responsive to the piston moving within the cylinder in the second direction.

7. The method of claim 6, wherein returning the aspirated contents further comprises:
opening the first control valve, the third control valve, and the sixth control valve responsive to the piston moving within the cylinder in the first direction;
closing the second control valve, the fourth control valve, the fifth control valve and the seventh control valve responsive to the piston moving within the cylinder in the first direction;
opening the second control valve and the fifth control valve responsive to the piston moving within the cylinder in the second direction; and
closing the third control valve and the sixth control valve responsive to the piston moving within the cylinder in the second direction.

8. The method of claim 7, wherein delivering medical/nutritious fluids into the patient's stomach comprises pumping the medical/nutritious fluids from the feeding bag into the patient's stomach via the nasogastric tube, the medical/nutritious fluids comprising nutrients, medications, and combinations thereof

9. The method of claim 7, further comprising filling an entire internal volume of the nasogastric tube with water before aspirating the patient's stomach, filling an entire internal volume of the nasogastric tube comprising pumping water from a water source into the nasogastric tube, the water source connected in fluid communication with the hollow cylinder, the hollow cylinder further comprising a fifth port located on the first end in fluid communication with the inner volume of the first chamber, the fifth port connected in fluid communication with the water source, the fluid communication between the water source and the fifth port intercepted by a seventh control valve, pumping water from the water source into the nasogastric tube comprising:
closing the third control valve, the fourth control vale, the fifth control valve, and the sixth control valve responsive to the piston moving within the cylinder in the first direction;
opening the first control valve, the second control valve, and the seventh control valve responsive to the piston moving within the cylinder in the first direction;
closing the seventh control valve responsive to the piston moving within the cylinder in the second direction; and
opening the fifth control valve responsive to the piston moving within the cylinder in the second direction.

10. The method of claim 9, wherein each of the first control valve, the second control valve, the third control valve, the fourth control valve, the fifth control valve, the sixth control valve, and the seventh control valve comprise:
a respective motor;
a respective cam coupled to the respective motor via a respective shaft, the respective cam comprising a respective main body and a respective extended lip protruding from the respective main body along a plane perpendicular to a longitudinal axis of the respective shaft, the respective motor configured to drive a rotational movement of the respective cam about the longitudinal axis of the respective shaft; and
a respective seat mounted adjacent the respective cam with a gap between the respective seat and the respective main body of the respective cam along an axis perpendicular to the longitudinal axis of the respective shaft,
wherein closing each of the first control valve, the second control valve, the third control valve, the fourth control valve, the fifth control valve, the sixth control valve, and the seventh control valve comprises rotating the corresponding cam into a first rotational position, wherein the extended lip is positioned between the main body of the cam and the seat along the axis perpendicular to the longitudinal axis of the shaft closing the gap between the seat and the main body.

11. The method of claim 10, wherein opening each of the first control valve, the second control valve, the third control valve, the fourth control valve, the fifth control valve, the sixth control valve, and the seventh control valve comprises rotating the corresponding cam into a second rotational position, wherein the extended lip is not positioned between the main body of the cam and the seat along the axis perpendicular to the longitudinal axis of the shaft.

12. The method of claim 11, wherein connecting the nasogastric tube to the pumping mechanism comprises:
connecting the nasogastric tube to the second port via a first flexible tube, the first flexible tube positioned in the gap between the seat and the cam of the fifth control valve; and
connecting the nasogastric tube to the fourth port via a second flexible tube, the second flexible tube positioned in the gap between the seat and the cam of the sixth control valve.

13. The method of claim 12, wherein aspirating the patient's stomach comprises pumping out contents of the patient's stomach via the nasogastric tube and collecting the aspirated content of the patient's stomach within the collection bag, the collection bag connected to the third port via a third flexible tube, a first portion of the third flexible tube positioned in the gap between the seat and the cam of the first control valve, a second portion of the third flexible tube positioned in the gap between the seat and the cam of the second control valve.

14. The method of claim 13, wherein delivering fluids into the patient's stomach comprises pumping the fluids from the feeding bag into the patient's stomach via the nasogastric tube, the feeding bag connected to the first port via a fourth flexible tube, a first portion of the fourth flexible tube positioned in the gap between the seat and the cam of the fourth control valve, a second portion of the fourth flexible tube positioned in the gap between the seat and the cam of the third control valve.

15. A system for gastrointestinal feeding and aspiration, the system comprising:
a double action piston pump, comprising:
a hollow cylinder comprising a first end, a second end, and an annular wall extended between the first end and the second end along a longitudinal axis of the cylinder, the hollow cylinder further comprising a first port and a second port connected in fluid communication with an inner volume of the cylinder on the annular wall adjacent the first end, the hollow cylinder further comprising a third port and a fourth port connected in fluid communication with the inner volume of the cylinder on the annular wall adjacent the second end; and
a piston disposed within the cylinder, the piston dividing the inner volume of the cylinder into a first chamber and a second chamber, the piston reciprocating along the longitudinal axis between the first end and the second end, the first port and the second port located in the first chamber, the third port and the fourth port located in the second chamber;
a collection bag connected in fluid communication with the first port and the third port, the fluid communication between the collection bag and the first port intercepted by a first control valve and a second control valve, the fluid communication between the collection bag and the third port intercepted by the first control valve and a third control valve, the second control valve connected in series with the first control valve, the third control valve connected in series with the first control valve;
a feeding bag connected in fluid communication with the first port and the third port, the fluid communication between the feeding bag and the first port intercepted by a fourth control valve and the second control valve, the fluid communication between the feeding bag and the third port intercepted by the fourth control valve and the second control valve, the second control valve connected in series with the fourth control valve, the third control valve connected in series with the fourth control valve;
a nasogastric tube connected in fluid communication with the second port and the fourth port, the fluid communication between the nasogastric tube and the second port intercepted by a fifth control valve, the fluid communication between the nasogastric tube and the fourth port intercepted by a sixth control valve; and
a controller connected to the first control valve, the second control valve, the third control valve, the fourth control valve, the fifth control valve, and the sixth control valve, the controller configured to send opening/closing commands to actuators of the first control valve, the second control valve, the third control valve, the fourth control valve, the fifth control valve, and the sixth control valve.

16. The system of claim 14, wherein the hollow cylinder further comprises a fifth port located on the first end in fluid communication with the inner volume of the first chamber, the fifth port connected in fluid communication with a water source, the fluid communication between the water source and the fifth port intercepted by a seventh control valve, the controller further coupled with the seventh control valve, the controller further configured to send opening/closing commands to an actuator of the seventh control valve.

17. The system of claim 14, wherein each of the first control valve, the second control valve, the third control valve, the fourth control valve, the fifth control valve, the sixth control valve, and the seventh control valve comprise:
a respective motor;
a respective cam coupled to the respective motor via a respective shaft, the respective cam comprising a respective main body and a respective extended lip protruding from the respective main body along a plane perpendicular to a longitudinal axis of the respective shaft, the respective motor configured to drive a rotational movement of the respective cam about the longitudinal axis of the respective shaft in response to the opening/closing commands received from the controller; and
a respective seat mounted adjacent the respective cam with a gap between the respective seat and the respective main body of the respective cam along an axis perpendicular to the longitudinal axis of the respective shaft,
wherein the respective motor is further configured to drive the rotational movement of the respective cam between a first rotational position and a second rotational position, wherein the first rotational position corresponds to a position where the respective extended lip is positioned between the respective main body of the respective cam and the respective seat along the axis perpendicular to the longitudinal axis of the respective shaft closing the gap between the respective seat and the respective main body, wherein the second rotational position corresponds to a position where the respective extended lip is not positioned between the respective main body of the respective cam and the respective seat along the axis perpendicular to the longitudinal axis of the respective shaft,
wherein the respective motor is further configured to drive the rotational movement of the respective cam from the first rotational position to the second rotational position in response to an opening command received from the controller, and
wherein the respective motor is further configured to drive the rotational movement of the respective cam from the second rotational position to the first rotational position in response to a closing command received from the controller.

18. The system of claim 16, wherein:
the feeding bag is connected to the first port via a fourth flexible tube, a first portion of the fourth flexible tube positioned in the gap between the seat and the cam of the fourth control valve, a second portion of the fourth flexible tube positioned in the gap between the seat and the cam of the third control valve, and
the collection bag is connected to the third port via a third flexible tube, a first portion of the third flexible tube positioned in the gap between the seat and the cam of the first control valve, a second portion of the third flexible tube positioned in the gap between the seat and the cam of the second control valve.

19. The system of claim 17, wherein:
the nasogastric tube is connected to the second port via a first flexible tube, the first flexible tube positioned in the gap between the seat and the cam of the fifth control valve, and
the nasogastric tube is further connected to the fourth port via a second flexible tube, the second flexible tube positioned in the gap between the seat and the cam of the sixth control valve.

20. The system of claim 18, wherein the third flexible tube and the fourth flexible tube are interconnected via an interconnect tube, a first end of the interconnect tube connected in fluid communication with the third flexible tube at a point along the third flexible tube between the first control valve and the second control valve, a second opposite end of the interconnect tube connected in fluid communication with the fourth flexible tube at a point along the fourth flexible tube between the fourth control valve and the third control valve.

21. The system of claim 19, wherein the first control valve and the fourth control valve comprise a single double action valve intercepting both the first portion of the third flexible tube and the first portion of the fourth flexible tube, the single double action valve comprising:
a respective motor;
a respective cam coupled to the respective motor via a respective shaft, the respective cam comprising a respective main body and a respective first extended lip and a respective second extended lip protruding from the main body along a plane perpendicular to a longitudinal axis of the respective shaft, the respective first extended lip and the respective second extended lip attached to opposite sides of the respective main body along an axis perpendicular to the longitudinal axis of the respective shaft, the respective motor configured to drive a rotational movement of the respective cam about the longitudinal axis of the shaft in response to the opening/closing commands received from the controller;
a respective first seat mounted on a first side of the respective cam with a gap between the respective seat and the respective main body of the respective cam along the axis perpendicular to the longitudinal axis of the respective shaft; and
a respective second seat mounted on a second opposite side of the respective cam with a gap between the respective seat and the respective main body of the respective cam along the axis perpendicular to the longitudinal axis of the respective shaft,
wherein the first portion of the third flexible tube is positioned between the respective first seat and the first side of the respective main body, and the first portion of the fourth flexible tube is positioned between the respective second seat and the second opposite side of the respective main body, and
wherein the respective motor is further configured to drive the rotational movement of the respective cam about the longitudinal axis of the respective shaft from an initial position between a first position and a second position, the initial position corresponding to a position where the respective first extended lip is positioned adjacent an outer surface of the first portion of the third flexible tube and the respective second extended lip is positioned adjacent an outer surface of the first portion of the fourth flexible tube, the first position corresponding to a position where the respective first extended lip is positioned between the respective first seat and the respective main body pressing shut the first portion of the third flexible tube, the second position corresponding to a position where the respective second extended lip is positioned between the respective second seat and the respective main body pressing shut the first portion of the fourth flexible tube.
